(19) United States
(12) Patent Application Publication
(10) Pub. No.: US 2025/0387030 A1
(43) Pub. Date: Dec. 25, 2025

(54) BIOMETRIC MEASUREMENT DEVICE AND SYSTEM

(71) Applicant: VMEDMOBILE LLC, Town and Country, MO (US)

(72) Inventors: Eric Preston Thomas, North Ogdan, UT (US); Patrick James O'Brien, St. Louis, MO (US); Donald Eric Hendrickson, Simi Valley, CA (US)

(21) Appl. No.: 19/248,044

(22) Filed: Jun. 24, 2025

Related U.S. Application Data
(60) Provisional application No. 63/663,594, filed on Jun. 24, 2024.
(60) Provisional application No. 63/663,624, filed on Jun. 24, 2024.
(60) Provisional application No. 63/663,616, filed on Jun. 24, 2024.
(60) Provisional application No. 63/663,628, filed on Jun. 24, 2024.

(51) Int. Cl. A61B 5/0205; A61B 5/00; A61B 5/021; A61B 5/024; A61B 5/1455; A61B 5/318

(52) U.S. Cl. CPC A61B 5/02055; A61B 5/0006; A61B 5/0008; A61B 5/002; A61B 5/14551; A61B 5/318; A61B 5/681; A61B 5/6898; A61B 5/742; A61B 5/7475; A61B 5/021; A61B 5/02438

(57) ABSTRACT

A system, comprising: a device that comprises: a case, a human body interface, a plurality of sensor modules, each coupled with the body interface, and each configured to detect a different biometric signal or indicator, a short range wireless communication interface, configured to communicate with a mobile device automatically, without the need to pair to the mobile device, at least one hardware processor, and device software configured to communicate the detected biometric signals or indicators to the mobile device via the short range wireless communication interface; and wherein the mobile device comprises: a user interface, a mobile device, short range wireless communication interface, configured to communicate with the device automatically, without the need to pair to the mobile device; and mobile device software configured to display via the user interface information based on the biometric signals received from the device via the mobile device, short range wireless communication interface.

### CROSS-REFERENCE TO RELATED APPLICATIONS

This application claims priority to U.S. Provisional Application No. 63/663,594, entitled, “Biometric Measurement Device and System” filed Jun. 24, 2024; U.S. Provisional Application No. 63/663,624, entitled, “Biometric Measurement Device and Marketplace”, filed Jun. 24, 2024; U.S. Provisional Application No. 63/663,616, entitled “Biometric Measurement Device and Platform, filed Jun. 24, 2024; U.S. Provisional Application No. 63/663,628, entitled “Biometric Measurement Device”, filed Jun. 24, 2024 and is related to U.S. patent application Ser. No. 17/493,821 (the '821 application), entitled “Smartphone Cover with Sensor Methods,” filed Oct. 4, 2021, which claims priority to U.S. Provisional Patent App. No. 63/086,950, filed on Oct. 2, 2020, each of which are hereby incorporated herein by reference as if set forth in full.

### BACKGROUND

### Field of the Invention

The embodiments described herein are generally directed to biometric sensing, and more particularly to systems and methods for sensing multiple biometrics using multiple modalities incorporated into a single device.

### Description of the Related Art

There are numerous biometric measuring devices on the market. Many of these are wearable devices that use for health and wellness applications. Pedometers, heart rate monitors, blood pressure cuffs, wireless scales, etc., are becoming ubiquitous, and now many such devices can actually capture and electrocardiogram (ECG/EKG). More and more, these wearable devices are incorporating a plurality of such modalities, or capabilities to measure these various biometrics. Almost all such devices are “paired” with some for of platform that comprises a backend and/or an application on the user's wearable device or on a mobile device of the user.

The problem with these platforms is that they tend to be specific to providing certain information in certain formats. There are few if any platforms that attempt to look at all such information holistically and provide health information that may be indicative of overall health. For example, a device(s)/platform may be able to acquire step information and heart rate information and may convey, e.g., through an application on the user's device, the number of steps, heart rate, and calories burned during a particular time period, i.e., a work-out, or daily, etc. But if the user is tracking all this information in order to lose weight, such a hypothetical platform/application often do not track weight. Thus, the user has to get another application, and possibly pay a fee, in order to track their weight, and e.g., calorie intake. Thus, a user may end up with a plurality of devices and applications/platforms, and subscriptions associated therewith, in order to track and correlate the information they desire.

Moreover, while telehealth services are increasing, thanks in part to relaxed regulatory frameworks and easier reimbursement, these services rarely tap into or make use of the type of biometric information that can be gathered by such devices. Users can potentially forward such information to their doctor, but such a processes are ad-hoc at best. Thus, healthcare services fail to take advantage of this vast amount of information that is being gathered by these devices.

### SUMMARY

Accordingly, systems, methods, devices and non-transitory computer-readable media are disclosed to sense multiple biometrics using multiple modalities incorporated into a single device.

According to one aspect, a system, comprises a device that comprises: a case, a human body interface, a plurality of sensor modules, each coupled with the body interface, and each configured to detect a different biometric signal or indicator, a short range wireless communication interface, configured to communicate with a mobile device automatically, without the need to pair to the mobile device, at least one hardware processor, and device software that is configured to, when executed by the at least one hardware processor, communicate the detected biometric signals or indicators to the mobile device via the short range wireless communication interface; and wherein the mobile device comprises: a user interface, a mobile device, short range wireless communication interface, configured to communicate with the device automatically, without the need to pair to the mobile device; and mobile device software that is configured to, when executed by the at least one hardware processor, display via the user interface information based on the biometric signals received from the device via the mobile device, short range wireless communication interface.

It should be understood that any of the features in the methods above may be implemented individually or with any subset of the other features in any combination. Thus, to the extent that the appended claims would suggest particular dependencies between features, disclosed embodiments are not limited to these particular dependencies. Rather, any of the features described herein may be combined with any other feature described herein, or implemented without any one or more other features described herein, in any combination of features whatsoever. In addition, any of the methods, described above and elsewhere herein, may be embodied, individually or in any combination, in executable software modules of a processor-based system, such as a server, and/or in executable instructions stored in a non-transitory computer-readable medium.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment;

FIGS. 3A and B illustrate a smart phone cover implementation of a multi-model measurement device configured in accordance with one example embodiment;

FIG. 4 illustrates another smart phone cover implementation of a multi-model measurement device configured in accordance with one example embodiment;

FIG. 5 illustrates that a circuit can be included in the smart phone covers of FIGS. 3 and 4;

FIG. 6 illustrates the circuit of FIG. 5 in more detail, according to one example embodiment;

FIGS. 7A and B illustrate a tablet cover implementation of a multi-model measurement device configured in accordance with one example embodiment;

FIG. 8 illustrates a handheld implementation of a multi-model measurement device configured in accordance with one example embodiment;

FIG. 9 illustrates a watch implementation of a multi-modal biometric sensing device as described herein; and

FIG. 10 illustrates a processes for body characteristics using the systems and methods described herein.

### DETAILED DESCRIPTION

In an embodiment, systems, methods, devices and non-transitory computer-readable media are disclosed for sensing multiple biometrics using multiple modalities incorporated into a single device.

After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

### 1. System Overview

### 1.1. Infrastructure

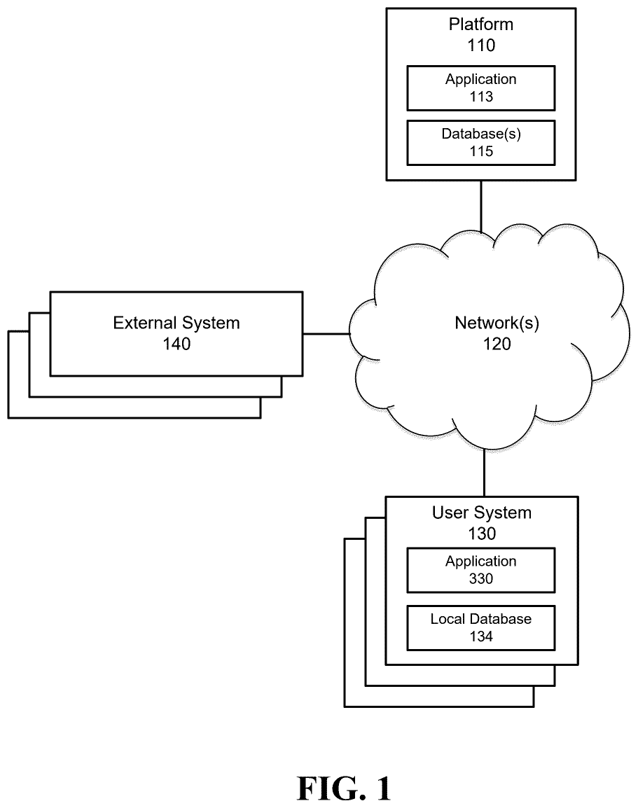

FIG. 1 illustrates an example infrastructure in which one or more of the disclosed processes may be implemented, according to an embodiment. The infrastructure may comprise a platform 110 (e.g., one or more servers) which hosts and/or executes one or more of the various processes (e.g., methods or functions, implemented as software modules) described herein. Platform 110 may comprise dedicated servers, or may instead be implemented in a computing cloud, in which the resources of one or more servers are dynamically and elastically allocated to multiple tenants based on demand. In either case, the servers may be collocated and/or geographically distributed. Platform 110 may also comprise or be communicatively connected to a server application 113 and/or one or more databases 115. In addition, platform 110 may be communicatively connected to one or more user systems 130 via one or more networks 120. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 113, and one set of database(s) 115 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 130 may comprise or be communicatively connected to a client application 132 and/or one or more local databases 134.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 115) that are locally and/or remotely accessible to platform 110. It should be understood that platform 110 may also respond to other requests from user system(s) 130.

Platform 110 may comprise, be communicatively coupled with, or otherwise have access to one or more database(s) 115. For example, platform 110 may comprise one or more database servers which manage one or more databases 115. Server application 113 executing on platform 110 and/or client application 132 executing on user system 130 may submit data (e.g., user data, form data, etc.) to be stored in database(s) 115, and/or request access to data stored in database(s) 115. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 113), executed by platform 110.

In embodiments in which a web service is provided, platform 110 may receive requests from user system(s) 130 and/or external system(s) 140, and provide responses in extensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes (e.g., methods and functionality), storage, and/or the like, described herein. For example, in such an embodiment, a client application 132, executing on one or more user system(s) 130, may interact with a server application 113 executing on platform 110 to execute one or more or a portion of one or more of the various process(es) described herein.

Client application 132 may be “thin,” in which case processing is primarily carried out server-side by server application 113 on platform 110. A basic example of a thin client application 132 is a browser application, which simply requests, receives, and renders webpages at user system(s) 130, while server application 113 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 113 on platform 110, at any point along this spectrum between “thin” and “thick,” depending on the design goals of the particular implementation. In any case, the software described herein, which may wholly reside on either platform 110 (e.g., in which case server application 113 performs all processing) or user system(s) 130 (e.g., in which case client application 132 performs all processing) or be distributed between platform 110 and user system(s) 130 (e.g., in which case server application 113 and client application 132 both perform processing), can comprise one or more executable software modules comprising instructions that implement one or more of the processes (e.g., methods or functions) described herein.

### 1.2. Example Processing Device

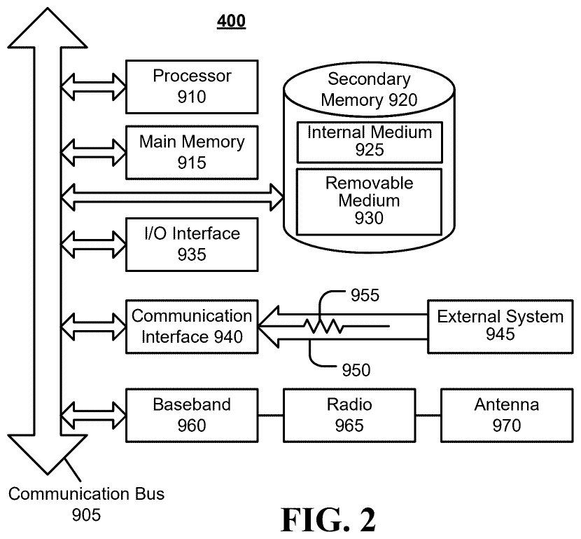

FIG. 2 is a block diagram illustrating an example wired or wireless system 900 that may be used in connection with various embodiments described herein. For example, system 900 may be used as or in conjunction with one or more of the processes (e.g., to store and/or execute the software), including any methods or functions, described herein, and may represent components of platform 110, user system(s) 130, external system(s) 140, and/or other processing devices described herein. System 900 can be any processor-enabled device (e.g., server, personal computer, etc.) that is capable of wired or wireless data communication. Other processing systems and/or architectures may also be used, as will be clear to those skilled in the art.

System 900 may comprise one or more processors 910. Processor(s) 910 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor (e.g., back-end processor), an additional microprocessor or controller for dual or multiple processor systems, and/or a coprocessor. Such auxiliary processors may be discrete processors or may be integrated with a main processor 910. Examples of processors which may be used with system 900 include, without limitation, any of the processors (e.g., Pentium™, Core i7™, Core i9™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor(s) 910 may be connected to a communication bus 905. Communication bus 905 may include a data channel for facilitating information transfer between storage and other peripheral components of system 900. Furthermore, communication bus 905 may provide a set of signals used for communication with processor 910, including a data bus, address bus, and/or control bus (not shown). Communication bus 905 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 900 may comprise main memory 915. Main memory 915 provides storage of instructions and data for programs executing on processor 910, such as any of the software discussed herein. It should be understood that programs stored in the memory and executed by processor 910 may be written and/or compiled according to any suitable language, including without limitation C/C++, Java, JavaScript, Perl, Python, Visual Basic, .NET, and the like. Main memory 915 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 900 may comprise secondary memory 920. Secondary memory 920 is a non-transitory computer-readable medium having computer-executable code and/or other data (e.g., any of the software disclosed herein) stored thereon. In this description, the term “computer-readable medium” is used to refer to any non-transitory computer-readable storage media used to provide computer-executable code and/or other data to or within system 900. The computer software stored on secondary memory 920 is read into main memory 915 for execution by processor 910. Secondary memory 920 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 920 may include an internal medium 925 and/or a removable medium 930. Internal medium 925 and removable medium 930 are read from and/or written to in any well-known manner. Internal medium 925 may comprise one or more hard disk drives, solid state drives, and/or the like. Removable storage medium 930 may be, for example, a magnetic tape drive, a compact disc (CD) drive, a digital versatile disc (DVD) drive, other optical drive, a flash memory drive, and/or the like.

System 900 may comprise an input/output (I/O) interface 935. I/O interface 935 provides an interface between one or more components of system 900 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing systems, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet computer, or other mobile device).

System 900 may comprise a communication interface 940. Communication interface 940 allows software to be transferred between system 900 and external devices (e.g. printers), networks, or other information sources. For example, computer-executable code and/or data may be transferred to system 900 from a network server (e.g., platform 110) via communication interface 940. Examples of communication interface 940 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 900 with a network (e.g., network(s) 120) or another computing device. Communication interface 940 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 940 is generally in the form of electrical communication signals 955. These signals 955 may be provided to communication interface 940 via a communication channel 950 between communication interface 940 and an external system 945 (e.g., which may correspond to an external system 140, an external computer-readable medium, and/or the like). In an embodiment, communication channel 950 may be a wired or wireless network (e.g., network(s) 120), or any variety of other communication links. Communication channel 950 carries signals 955 and can be implemented using a variety of wired or wireless communication means including wire or cable, fiber optics, conventional phone line, cellular phone link, wireless data communication link, radio frequency (“RF”) link, or infrared link, just to name a few.

Computer-executable code is stored in main memory 915 and/or secondary memory 920. Computer-executable code can also be received from an external system 945 via communication interface 940 and stored in main memory 915 and/or secondary memory 920. Such computer-executable code, when executed, enable system 900 to perform the various process(es) of the disclosed embodiments as described elsewhere herein.

In an embodiment that is implemented using software, the software may be stored on a computer-readable medium and initially loaded into system 900 by way of removable medium 930, I/O interface 935, or communication interface 940. In such an embodiment, the software is loaded into system 900 in the form of electrical communication signals 955. The software, when executed by processor 910, preferably causes processor 910 to perform one or more of the processes described elsewhere herein.

System 900 may comprise wireless communication components that facilitate wireless communication over a voice network and/or a data network (e.g., in the case of user system 130). The wireless communication components comprise an antenna system 970, a radio system 965, and a baseband system 960. In system 900, radio frequency (RF) signals are transmitted and received over the air by antenna system 970 under the management of radio system 965.

In an embodiment, antenna system 970 may comprise one or more antennae and one or more multiplexors (not shown) that perform a switching function to provide antenna system 970 with transmit and receive signal paths. In the receive path, received RF signals can be coupled from a multiplexor to a low noise amplifier (not shown) that amplifies the received RF signal and sends the amplified signal to radio system 965.

In an alternative embodiment, radio system 965 may comprise one or more radios that are configured to communicate over various frequencies. In an embodiment, radio system 965 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 965 to baseband system 960.

If the received signal contains audio information, then baseband system 960 decodes the signal and converts it to an analog signal. Then the signal is amplified and sent to a speaker. Baseband system 960 also receives analog audio signals from a microphone. These analog audio signals are converted to digital signals and encoded by baseband system 960. Baseband system 960 also encodes the digital signals for transmission and generates a baseband transmit audio signal that is routed to the modulator portion of radio system 965. The modulator mixes the baseband transmit audio signal with an RF carrier signal, generating an RF transmit signal that is routed to antenna system 970 and may pass through a power amplifier (not shown). The power amplifier amplifies the RF transmit signal and routes it to antenna system 970, where the signal is switched to the antenna port for transmission.

Baseband system 960 is communicatively coupled with processor(s) 910, which have access to memory 915 and 920. Thus, software can be received from baseband processor 960 and stored in main memory 910 or in secondary memory 920, or executed upon receipt. Such software, when executed, can enable system 900 to perform the various process(es) of the disclosed embodiments.

### 1.3. Example Devices

The following are several example embodiments for a multi-modal, biometric sensing device configured in accordance with systems and methods described herein.

### 1.3.1 Smart Phone Cover

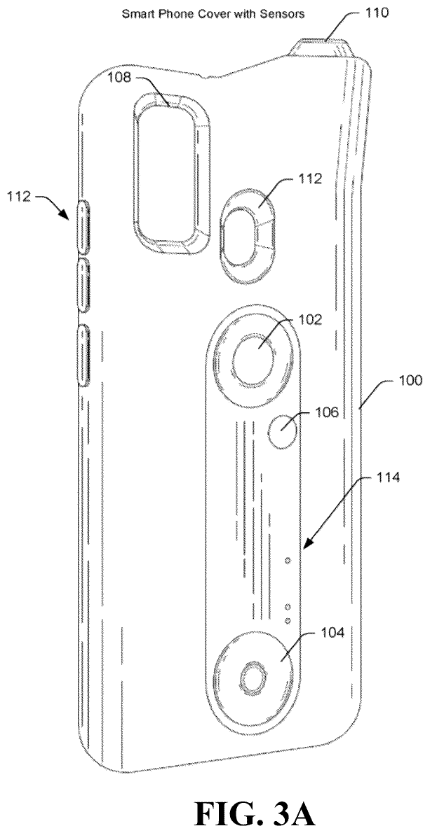

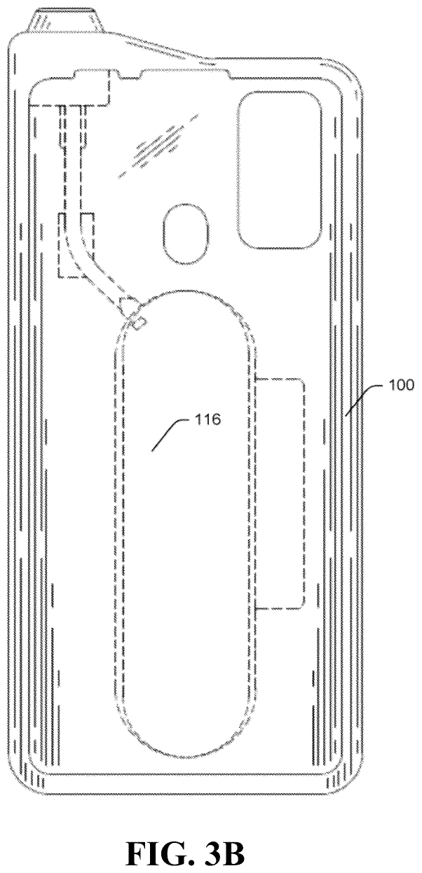

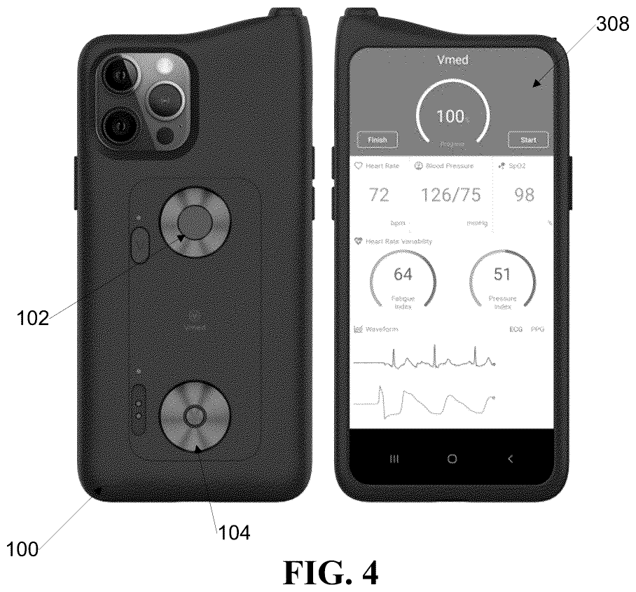

FIGS. 1-5 of the '821 application describe a smart phone cover implementation of a multi-model measurement device 100 configured in accordance with one example embodiment. In other words, the device 100 of FIG. 1A of the '821 application (recreated here as FIG. 3), can be configured to physically and communicatively integrated with a smartphone that is acting as a user system 130 in the architecture of FIG. 1. Referring to FIGS. 1A-1 B (3A-3B herein), there is shown a smartphone cover 100 for at least partially enclosing a smart mobile communications device (also referred to as a smartphone and is not shown). The cover 100 is molded to cover the back and sides of any smartphone while allowing the display 308 (see FIG. 4) of the smartphone to be visible and unobstructed at least partially. The mobile communications device can be any communications device having a display 308 and a communications channel, including but not limited to, a smartphone, an iPhone, an Android based phone, an iPad, a PDA, a personal communications device, and a personal assistant device.

The smartphone cover 100 can include medical module pads 102 and 104, inferred thermometer/power on button 106, camera aperture 108, fingerprint reader aperture 112, indicator LED's 114, inferred thermometer probe 110 and apertures for phone keys 112. Pads 102 and 104 can be used to detect at least some combination of a user's core body temperature (IRT), Blood Oxygen level (Sp02) heart rate, ECG and Blood Pressure. Infrared thermometer probe 110 can detect a user's core body temperature.

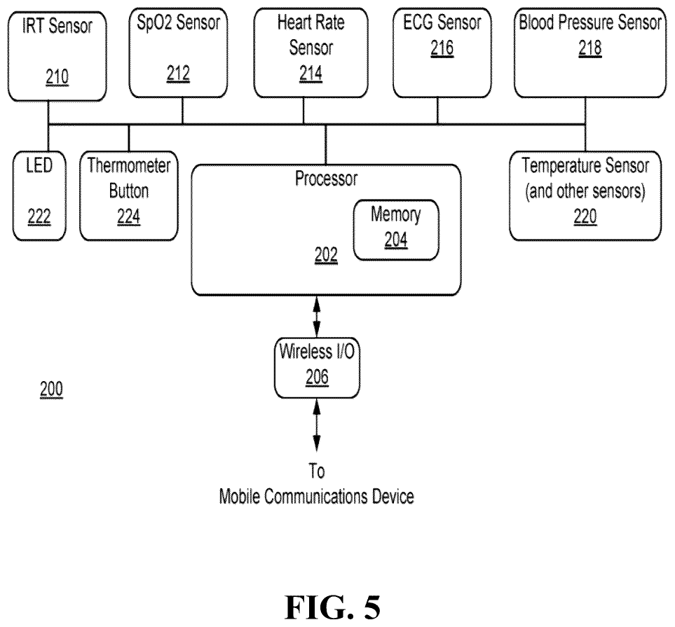

Referring to FIG. 3B, a circuit 116 (See FIG. 2 of the '182 application recreated herein as FIG. 5) can be integrally incorporated into cover 100. Circuit 116 can include a battery (not shown) and processor. In other embodiments, circuit 100 can be configured to receive battery power from the smart phone device. Circuit 116 can be electrically coupled with medical module pads 102 and 104, inferred thermometer button 106, indicator LED's 114, inferred thermometer probe 110.

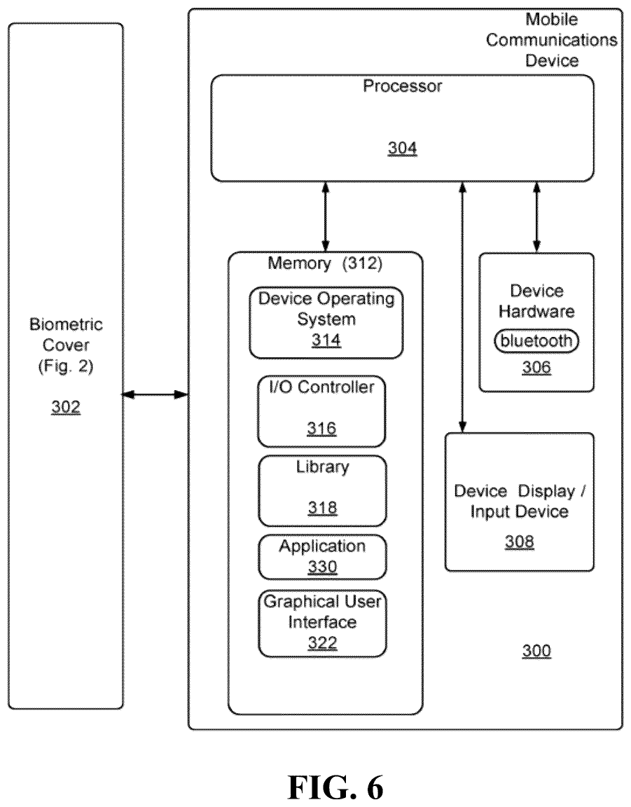

Referring to FIG. 5 herein (FIG. 2 of the '821 application), there is shown a simplified schematic diagram of a circuit 200 (which can be included in circuit 116 in FIG. 4) for monitoring a user's health/medical characteristics. Circuit 200 include a processor 202, which can be a processor 910, having a memory 204, which can be main memory 915, for storing program instructions other code for executing the processes shown or describe in connection with FIG. 6 (FIG. 4 in the '821 application). Processor 202 can be coupled with a wireless (or wireline) Input/Output (I/O) device (which can be I/O Interface 935) such as a Bluetooth communications device 206. Processor 202 can, for example, be coupled with IRT Sensor 210, Sp02 sensor 212, heart rate sensor 214, EKG sensor 216, blood pressure sensor 218, and temperature sensor 220, blood sugar sensor (not shown), and breathing rate sensor or respiratory rate (breathing rate) sensor (not shown). The sensors can be separate devices within cover 100 or can be implemented as single sensor device, such as medical module pads 102 and 104, in combination with processor 202 running software or firmware instructions stored in memory 204. Processor 202 can be coupled with light emitting diodes 222 and thermometer and/or medical module power on button 224 (Button 106 of FIG. 3A).

In certain embodiments, with the various sensor inputs, the following measurement can then be made using platform 110 and/or application 330. As described in more detail below, these measurements can then be displayed to the user via graphical user interface 322 and/or provided to a physician for use in a telehealth follow up.

### Cardiovascular Measurements

Electrocardiogram (ECG/EKG): An electrocardiogram measures the electrical impulses of the heart to create a graph showing how your heart is beating. Physicians use this graph as a measure of overall cardiac health and performance.

Afibrillation/Arrythmia (AFIB): The Vmed device measures and records a two-lead EKG. This EKG contains enough data to identify afribrillation (AFIB), one of the most common—and deadly—forms of cardiac arrythmias. AFIB is a leading cause of strokes.

Heart Rate: Heart rate of pulse is one of the key indicators of overall health and wellness, as well as cardiac health. Generally speaking, the lower the resting pulse, the better a patient's health. Device 302 measures and tracks a patient's pulse over time.

Heart Rate Variability (HRV): Heart rate variability measures the time between beats of the heart. For some cardiac patients, this metric can be a vital tool in monitoring and improving cardiac health.

Blood Pressure (BP): Blood pressure is one of the most common and important cardiac health indicators. Device 302 can be calibrated to take highly precise measurements and track them over time, providing the patient and the physician with a picture of overall BP patterns to diagnose and treat hypertension and other conditions.

### Pulmonary Measurements

Blood Oxygen (Sp02): Blood oxygen levels show how saturated the blood is with oxygen, reporting a measurement as a percentage. Individuals with normal saturation will see between 96% and 99%, while individuals suffering from conditions like COPD or other pulmonary conditions may experience measurements that are lower. Device 302 tracks PSO2 so the physician can gauge how well your lungs function.

Respiration Rate: The rate of inhaling and exhaling can demonstrate overall pulmonary function. Over time, it's natural for respiration rate to vary significantly. However, the level of this variation can tell the physician how healthy both the heart and lungs are.

### General Health Telemetry

Temperature: Temperature can be a leading indicator of infection, and device 302 can measure and track temperature, which can then be combined with other measurements to detect health conditions as described in more detail below.

Referring to FIG. 6 (FIG. 3 of the '821 application), there are illustrated selected modules in smartphone 300 (also referred to herein as a mobile communications device). Smartphone 300 communicates with device 302, which can be cover 100, or as explained below can be another form of multi-modal biometric sensing device, and that can include circuit 116. Smartphone 300 includes a processing device 304 (which can be a processor 910), memory 312 (which can be a memory 915), and display/input device 308. Processing device 304 can include a microprocessor, microcontroller or any such device for accessing memory 312 and display/input device 308. Processing device 304 has processing capabilities and memory suitable to store and execute computer-executable instructions. In one example, processing device 304 includes one or more processors.

Processing device 304 executes instructions stored in memory 312, and in response thereto, processes signals from and to display/input device 308 and device hardware 306 which may include a clock/timer. Device hardware 306 may include input device, network 1/o device (not shown) that includes network and communication circuitry (e.g. Bluetooth circuitry, near field communications, and wifi, etc.) for communicating with a communications network and output device 329 for communicating with a wireless I/O device 206 in cover 200 (FIG. 5). Input device 308 receives inputs from a user of the personal computing device and may include a keyboard, mouse, track pad, microphone, audio input device, video input device, or touch screen display. Display device 308 may include an LED, LCD, CRT, or any type of display screen.

Memory 312 (and memory 204) can include a non-transitory volatile and nonvolatile memory, removable and non-removable media implemented in any method or technology for storage of information, such as computer-readable instructions, data structures, program modules or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium (including a non-transitory computer readable storage medium) which can be used to store the desired information, and which can be accessed by a computer system.

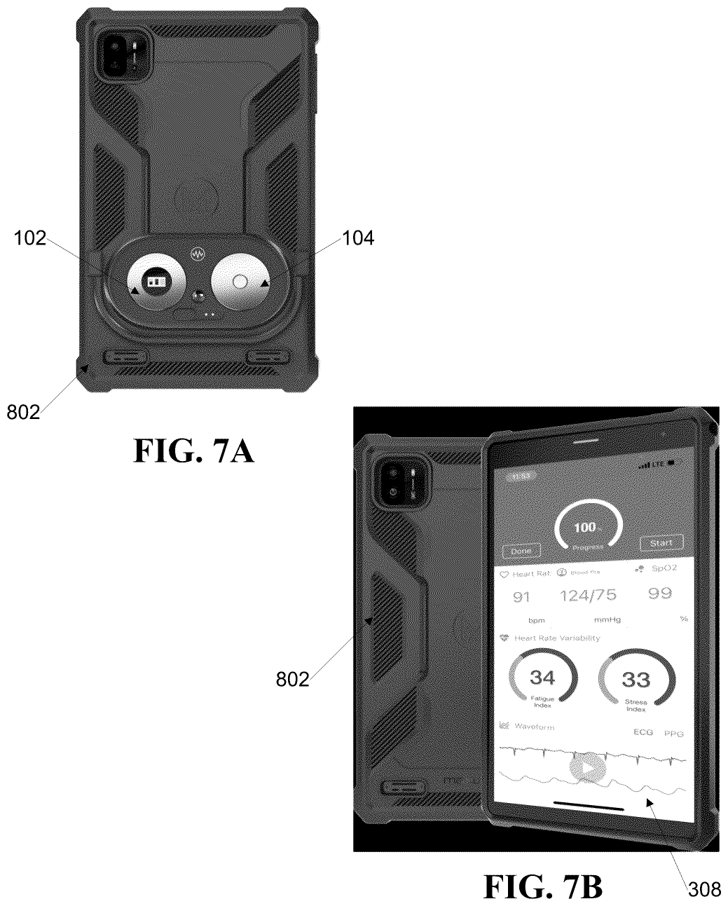

Modules stored in memory 312 of the computing device 208 can include an operating system 314, an I/O controller 316, a library 318, an application 330 and a graphical user interface 322 that can be displayed on display 308. Operating system 314 can be used by application 330 to operate display 308. Library 316 can include preconfigured parameters (or set by the user before or after initial operation) such as computing device operating parameters and configurations. Application 330 can include a body characteristic testing programs and other code for executing the processes shown or describe in connection with FIG. 7 (FIG. 5 of the '821 application).

### 1.3.2 Tablet Phone Cover

FIGS. 7A and 7B illustrate that the form of the device and cover is not necessarily critical as in this example the device is a tablet and the cover 802 is configured to interface with the device in a similar manner as cover 100. As can be seen, pads 102 and 104 can be integrated in cover 802 and a circuit 116 can be included in cover 802. Also, as can be seen the device can comprise a display 308 that can include a user interface 322 that can be used by an application 330 to display information related to the biometrics signals being sensed and processed by processor 202 and application 330. What information can be displayed and how it is generated is described in more detail below.

### 1.3.3 Hand Held Device

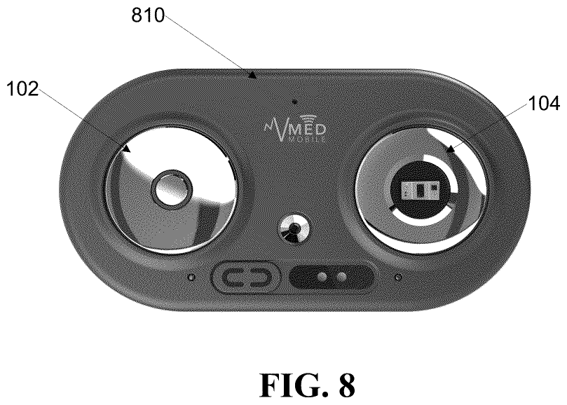

FIG. 8 illustrates a hand held implementation of a multi-modal biometric sensing device 810 in accordance with one example embodiment. Device 810 can be constructed such that it is “palm-sized.” As can be seen, device 810 can comprise pads 102 and 104, as well as a circuit 119.

It should be noted, as mentioned above that covers 100 and 802, as well as device 810 can interface with the smart phone, tablet, etc., i.e., device 300 via wireless I/O 206. For example, as mentioned, short range wireless communication protocols such as Bluetooth™ can be used to interface with device 300. It should be understood, however that using such communication protocols requires some kind of pairing between, e.g., device 810 and device 300. This pairing typically requires the user of device 300 to actively perform a pairing operation. Unfortunately, the requirement is an impediment to an significant portion of users that will result in those users not using the device 810, in this case.

Fortunately, in accordance with the systems and methods described herein, user devices 300 can be configured remotely ahead of time such that when covers 100, 802 or device 810 are turned on in the vicinity of the user's device 300 they are already connected to the device 300 and can start communicating data to application 330. This “autopairing” is described in U.S. Pat. Nos. 10,701,746, 11,418,939, 11,419,168, and 11,785,438, which are each incorporated by reference herein as if set forth in full.

As such the adoption and use of covers 100 and 802 and/or device 810 is markedly higher than for other biometric sensing devices.

### 1.3.4 Watch Implementation

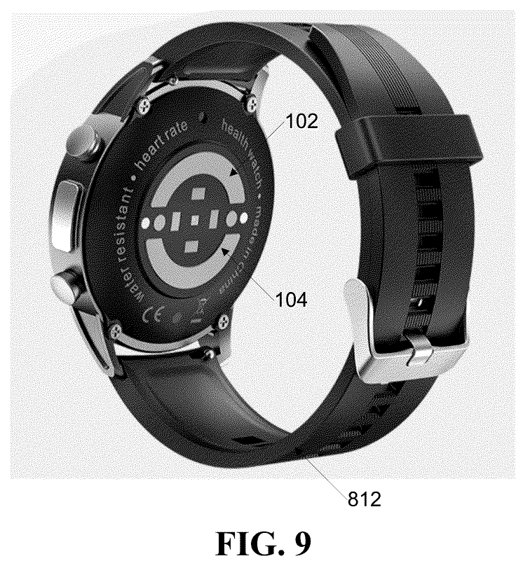

FIG. 9 illustrates a watch 812 implementation of a multi-modal biometric sensing device as described herein. But it should be understood that such a device as described herein can be incorporated into any type of wearable device.

### 2. Process Overview

Embodiments of processes for sensing multiple biometrics using multiple modalities incorporated into a single device will now be described in detail. It should be understood that the described processes may be embodied in one or more software modules that are executed by one or more hardware processors (e.g., processor 210), for example, as a software application (e.g., server application 113, client application 132, and/or a distributed application comprising both server application 113 and client application 132), which may be executed wholly by processor(s) of platform 110, wholly by processor(s) of user system(s) 130, or may be distributed across platform 110 and user system(s) 130, such that some portions or modules of the software application are executed by platform 110 and other portions or modules of the software application are executed by user system(s) 130. The described processes may be implemented as instructions represented in source code, object code, and/or machine code. These instructions may be executed directly by hardware processor(s) 210, or alternatively, may be executed by a virtual machine operating between the object code and hardware processor(s) 210. In addition, the disclosed software may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

### 2.1. Obtaining Biometrics

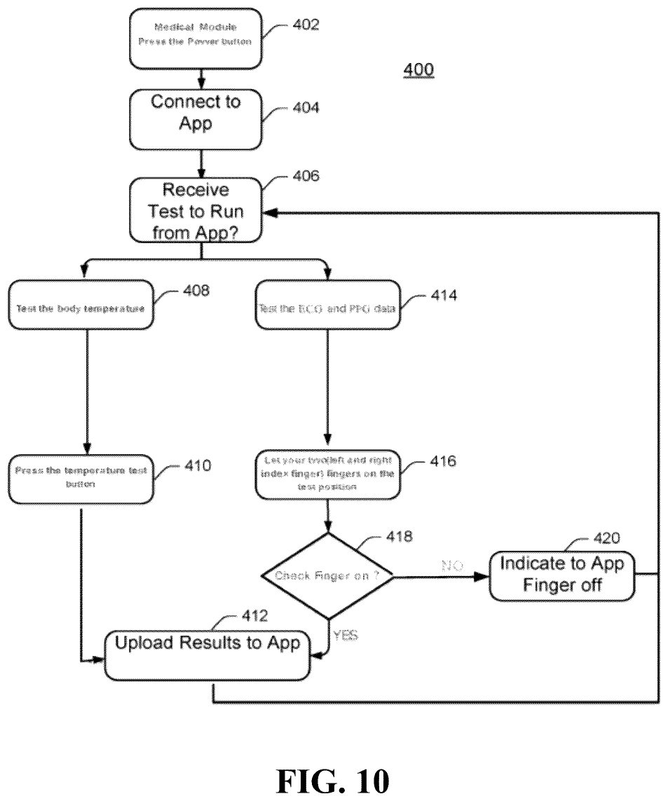

Illustrated in FIG. 10 (FIG. 4 of the '821 application), there is shown processes 400 for body characteristics using the systems and methods described herein. The exemplary processes in FIGS. 10 and 11 are illustrated as a collection of steps in a logical flow diagram, which represents a sequence of operations that can be implemented in hardware, software, and a combination thereof. In the context of software, the steps represent computer-executable instructions that, when executed by one or more processors, perform the recited operations. Generally, computer-executable instructions include routines, programs, objects, components, data structures, and the like that perform functions or implement particular abstract data types. The order in which the operations are described is not intended to be construed as a limitation, and any number of the described blocks can be combined in any order and/or in parallel to implement the process.

Referring to FIG. 10, process 400 is shown for determining body characteristics of a subject using circuit 200, application 330, and/or platform 110. In the processes, the subjects body characteristics are measured via devices 210-224 with processor 202 in steps 402-420. The subjects body characteristics can include, but are not limited to, the user's temperature level, respiratory levels, blood pressure levels, blood oxygen level, IRT, Sp02, heart rate, and EKG.

In step 402, processor 202 detects that the user has pressed the medical module power button to turn device 302 on. Then, in step 404, in response to the power button being turned on, device 302 can connect to application 330 running on the smartphone or other device 300. Again, the processor 202 can connect to device 300 via Bluetooth or via any other communications protocol, including but not limited to Wi-Fi, cellular or wireline. Also, as noted above, the device 302 can be connected without the need to go through a pairing process and can immediately start communicating with application 330.

In block 406, processor 202 receives a signal from the App in smartphone 300 indicating which test to run. If the indication is to test body temperature, the process executes blocks 408-412. If the indication is an EKG or other test, the process executes block 412-420.

Thus, if device 302 is obtaining or testing the temperature, then in step 408, the processor 202 can initiate the body temperature test/measurement. Such initiation can include activating the temperature sensor 220. In step 410, the processor 202 can obtain the subject/users temperature in response to the user touching the thermometer probe 110 or possibly pads 102 and 104.

In step 412, the results can be communicated to application 330 or platform 110.

In step 414, processor 202 can obtain an ECG or other biometric information from one of the other sensors. In step 416, in response to the user, e.g., placing their fingers in the proper position on the pads 102 and 104, the processor 202 senses the biometrics as provided by the sensor. If processor 202 in step 418 determines that the finger was not or improperly placed on the sensor pads 102 and 104, an indication can be provided in step 420 to the user, e.g., either via an indicator (not shown) or by communicating to the application 330 that the finger was not properly on the pads 102 and 104, and application 330 can then generate some form of notification. Once the user's fingers are properly placed, then the desired biometrics can be obtained and communicated to application 330 or platform 110.

### 2.2. Biometric Analysis

Once the biometric information is obtained, application 330 and/or application 113 can store the information in local database 134 and/or database(s) 115. The biometric information can then be used, either alone, i.e., individual measurements, in combination, and or in combination with other information, e.g., obtained from external systems 140 to ascertain averages in the biometric information over time, trends in the biometric information, to generate alerts related to the biometric information, determine health metric and/or health issues, etc. It should also be noted that because platform 110 can obtain information across various populations, platform 110 can analyze trends across those populations and can be used to predict, e.g., health issues that may affect whole populations like epidemics or pandemics, spread of certain diseases, etc.

For example, insurance companies can use the information to see trends across their insured populations that may indicate increased risks, and can then determine cations based on the biometric data that may reduce those risks. Such risks could be regional, age related, co-morbidity related, some combination thereof, etc.

Thus, such an insurance company or other entity could provide all their members a device 302 and since the device 302 is automatically connected via the autopair feature to device 300 it should be easy for users to use, which should increase adoption. In fact, such entities can offer rewards, e.g., decreased premiums for using the device 302.

As noted above, the biometric measurements can be used to determine conditions such as Afib and HRV. Further, temperature of the user combined with e.g., heart rate can indicate a specific pathogen and/or physiological health issues in the human body. For example, when the detected temperature of the user exceeds 100 degrees Fahrenheit, the heart rate of the user exceeds 110% of the average user heart rate detected by the sensor over a predetermined period of time, the detected respiratory rate of the user has increased by more than 20% of normal, the detected Sp02 of the user drops 3% of normal or below 94% oxygen saturation, or some combination thereof, can be indicative of an illness such as the flu.

As noted below, users can access healthcare, like telehealth sessions through application 330 and/or platform 110. Thus, diagnosis can be combined with the biometric data, which can further allow platform 110 to see patterns in the biometric data. For example, if patients are experiencing certain changes in biometric data, like a temperature along with slightly elevated respiratory rate and/or a drop in Sp02 and these changes are highly correlative with a certain diagnosis, then platform 110 can be configured to start recognizing such a pattern in the data and alerting the patient/user or their doctor. Platform 110 can also detect acceleration or decelerations within populations of certain conditions based on the measurements and other data maintained within platform 110 or accessible, e.g., via external systems 140.

The display 308 (FIGS. 5 and 8) illustrate that biometric information can be presented by user interface 322 to the user. This information can include information such as heart rate, blood pressure, Sp02, ECG, PPG, etc. But in addition, condition information can be derived and displayed from the biometric data. As can be seen in the center of display 308 in FIG. 5, for example, heart rate variability can be displayed in the form of a fatigue index and pressure index.

As noted above the user interface 322 can also be used to present a healthcare marketplace that can offer the user access to services such as telehealth services and prescription services. Thus, the user can select a doctor and request that their biometric information shared or accessible to the selected doctor. An appointment can then be scheduled. Application 330 can also be configured such that the telehealth appointment can be carried out through the application 330. If a prescription is required, then the user can select a pharmacy through the application 330 via the marketplace to arrange delivery of the prescription, all through platform 110 and application 330/113.

In certain embodiments, platform 110 can also provide billing services for, e.g., the doctors and/or pharmacy services in the marketplace.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

As used herein, the terms “comprising,” “comprise,” and “comprises” are open-ended. For instance, “A comprises B” means that A may include either: (i) only B; or (ii) B in combination with one or a plurality, and potentially any number, of other components. In contrast, the terms “consisting of,” “consist of,” and “consists of” are closed-ended. For instance, “A consists of B” means that A only includes B with no other component in the same context.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A system, comprising:
a device that comprises:
a case,
a human body interface,
a plurality of sensor modules, each coupled with the body interface, and each configured to detect a different biometric signal or indicator,
a short range wireless communication interface, configured to communicate with a mobile device automatically, without the need to pair to the mobile device,
at least one hardware processor, and
device software that is configured to, when executed by the at least one hardware processor, communicate the detected biometric signals or indicators to the mobile device via the short range wireless communication interface; and
wherein the mobile device comprises:
a user interface,
a mobile device, short range wireless communication interface, configured to communicate with the device automatically, without the need to pair to the mobile device; and
mobile device software that is configured to, when executed by the at least one hardware processor, display via the user interface information based on the biometric signals received from the device via the mobile device, short range wireless communication interface.

2. The system of claim 1, wherein the plurality of sensor modules are configured to detect some combination of the following biometric signals or indicators: internal core temperature, blood oxygen level (SpO2), heart rate, Electrocardiogram (ECG), blood pressure, temperature.

3. The system of claim 1, wherein the plurality of sensor modules are configured to detect the following biometric signals or indicators: internal core temperature, blood oxygen level (SpO2), heart rate, Electrocardiogram (ECG), blood pressure, temperature.

4. The system of claim 2, wherein the device software is further configured to, when executed by the at least one hardware processor, determine at least one of the following respiratory rate, afibrillation (Afib), hypertension, and Heart Rate Variability (HRV).

5. The system of claim 3, wherein the device software is further configured to, when executed by the at least one hardware processor, determine at least one of the following respiratory rate, afibrillation (Afib), hypertension, and Heart Rate Variability (HRV).

6. The system of claim 2, wherein the device software that is further configured to, when executed by the at least one hardware processor, determine a health condition.

7. The system of claim 6, wherein the health condition is a viral infection, the flu, or COPD.

8. The system of claim 3, wherein the device software that is further configured to, when executed by the at least one hardware processor, determine a health condition.

9. The system of claim 8, wherein the health condition is a viral infection, the flu, or COPD.

10. The system of claim 1, wherein the case is oblong and fits in a typical person's palm.

11. The system of claim 1, wherein the human body interface comprises finger pads.

12. The system of claim 2, wherein the mobile device software is further configured to, when executed by the at least one hardware processor, determine at least one of the following respiratory rate, afibrillation (Afib), hypertension, and Heart Rate Variability (HRV).

13. The system of claim 3, wherein the mobile device software is further configured to, when executed by the at least one hardware processor, determine at least one of the following respiratory rate, afibrillation (Afib), hypertension, and Heart Rate Variability (HRV).

14. The System of claim 1, wherein the mobile device further comprises a long range wireless communication interface and wherein the mobile device software is further configured to, when executed by the at least one hardware processor, communicate the biometric signals or indicators to a platform.
